(19) United States
(12) Patent Application Publication
(10) Pub. No.: US 2003/0045582 A1
(43) Pub. Date: Mar. 6, 2003

(54) Method for treating hyperglycemia

(75) Inventors: Jean-Paul Wuerth, Quogue, NY (US); Kenneth Cartwright, Blooming Grove, NY (US)

Correspondence Address: Allen Bloom, Esq., Dechert Price & Rhoads, P.O. Box 5218, Princeton, NJ, 08543, US

(73) Assignee: Alteon, Inc.

(21) Appl. No.: 10/071,851

(22) Filed: Feb. 8, 2002

Related U.S. Application Data
(63) Continuation of application No. 09/876,874, filed on Jun. 7, 2001, now abandoned.
(60) Provisional application No. 60/210,114, filed on Jun. 7, 2000.

(51) Int. Cl. A61K 31/155

(52) U.S. Cl. 514/634

(57) ABSTRACT

Provided, among other things, are methods for treating mammals, such as humans, with diabetes mellitus to delay the onset of end stage renal disease, relating to administering an effective amount of a pharmaceutical composition, wherein said composition comprise, a compound selected from the group consisting of aminoguanidine, its pharmaceutically acceptable salts, and mixtures thereof. Methods are disclosed for the gradual administration of the compound for treatment of diabetes or other indications associated with damage caused by reducing sugars, for the use of a periodic screening test for crescentic glomerulonephritis, and for treating humans with indicia of overt diabetic nephropathy.

This application claims the priority of U.S. Provisional Application No. 60/210,114.

The present invention relates generally to the treatment of mammals such as humans with diabetes mellitus to delay the onset of end stage renal disease.

The reaction between glucose and proteins has been known for some time. One common manifestation is in the generation of brown pigments during the cooking of food. The involvement of sugar in this browning reaction was identified by Maillard in 1912, who observed that glucose or other reducing sugars react with amino acids to form adducts that undergo a series of dehydrations and rearrangements to form stable brown pigments. Further studies have suggested that stored and heat treated foods undergo nonenzymatic browning as a result of the reaction between glucose and the polypeptide chain, and that, as a result the proteins are cross-linked and exhibit decreased bioavailability.

This reaction between reducing sugars and food proteins was found to have its parallel in vivo. Thus, the nonenzymatic reaction between glucose and the free amino groups on proteins to form a stable adduct, known as the Amadori product, has been shown to occur with hemoglobin, wherein a rearrangement of the amino terminal of the beta-chain of hemoglobin by reaction with glucose, forms the adduct known as hemoglobin A1c. The reaction has also been found to occur with a variety of other body proteins, such as lens crystallins, collagen and nerve proteins. See Bucala et al., “Advanced Glycosylation; Chemistry, Biology, and Implications for Diabetes and Aging” in Advances in Pharmacology, Vol. 23, pp. 1-34, Academic Press (1992). The Maillard reaction also affects the kidney glomerular basement membranes, and these proteins deteriorate both with age and as a consequence of diabetes mellitus.

Protein cross-linking through advanced glycosylation product formation can decrease solubility of structural proteins such as collagen in vessel walls (see Brownlee et al., Science 232, pp. 1629-1632 (1986)), and can also trap serum proteins, such as lipoproteins to the collagen. Also, this can result in increased permeability of the endothelium and consequently covalent trapping of extravasated plasma proteins in subendothelial matrix, and reduction in susceptibility of both plasma and matrix proteins to physiologic degradation by enzymes. (See Brownlee et al., Diabetes, 35, Suppl. 1, p. 42A (1986)). For these reasons, the progressive occlusion of diabetic vessels induced by chronic hyperglycemia is believed to result, at least in part, from excessive formation of glucose-derived cross-links. Such diabetic macrovascular changes and microvascular occlusion can be effectively prevented by chemical inhibition of advanced glycosylation product formation utilizing a composition such as aminoguanidine, as disclosed in U.S. Pat. No. 5,262,152.

In U.S. Pat. Nos. 4,758,583; 5,100,919; 5,106,877; 5,130,324; 5,272,165; 5,612,332; 5,852,009, a method and associated agents were disclosed that served to inhibit the formation of advanced glycosylation endproducts by reacting with an early glycosylation product that results from the original reaction between the target protein and glucose. Such inhibition can serve as a treatment for diabetes (see, e.g., U.S. Pat. No. 5,262,152).

Studies indicate that the development of chronic diabetic damage in target organs is primarily linked to hyperglycemia so that tight metabolic control can delay or even prevent end-organ damage. See Nicholls et al., Lab. Invest. 60, No. 4, p. 486 (1989), which discusses the effects of islet isografting and aminoguanidine in murine diabetic nephrophathy. These studies further evidence that aminoguanidine diminishes aortic wall protein cross-linking in diabetic rats and confirm earlier studies by Brownlee et al., Science, 232, pp. 1629-1632 (1986) directed to additional target organ complications of diabetes. Also, an additional study showed the reduction of immunoglobulin trapping in the kidney by aminoguanidine (Brownlee et al., Diabetes, 35 Supple. 1, p. 42A (1986)).

Further evidence in the streptozotocin-diabetic rat model that aminoguanidine administration intervenes in the development of diabetic nephropathy was presented by Brownlee et al., Diabetes, 35, Suppl. 1, p. 42A (1986), with regard to morphologic changes in the kidney which are hallmarks of diabetic renal disease. These investigators reported that the increased glomerular basement membrane thickness, a major structural abnormality characteristic of diabetic renal disease, was prevented with aminoguanidine. See also Vlassara et al., Proc Natl. Acad. Sci., Vol. 91, pp. 11704-11708 (November 1994).

Taken together, these data indicate that inhibition and reversal of the formation of advanced glycosylation endproducts (AGEs), can prevent or delay, as well as to some extent reverse, late, as well as early, structural lesions due to diabetes, as well as changes during aging caused by the formation of AGEs. See Li et al., Proc. Natl. Acad. Sci., Vol. 93, pp. 3902-3907 (1996).

While the success that has been achieved with aminoguanidine and similar compounds is promising, a need continues to identify methods of treatment of diabetes mellitus and other indications associated with elevated sugar or damage caused by reducing sugars using aminoguanidine.

### SUMMARY OF THE INVENTION

The invention provides a method for, in a mammal such as a human, (i) improving the elasticity or reducing wrinkles of a skin, treating (ii) diabetes or treating or inhibiting the (iii) discoloration of teeth, or treating or preventing one or more of the following conditions: (iv) adverse sequelae of diabetes, (v) kidney damage, (vi) damage to blood vasculature (e.g., loss of elasticity), (vii) hypertension, (viii) retinopathy, (ix) damage to lens proteins, (x) cataracts, (xi) neuropathy or (xii) osteoarthritis, or (xiii) improving myocardial elasticity, or delaying (xiv) the onset of end stage renal disease, the method comprising administering to a subject an effective amount of a pharmaceutical composition, wherein said composition comprises a compound selected from the group consisting of aminoguanidine, its pharmaceutically acceptable salts, and mixtures thereof, wherein the compound is administered by gradual introduction over a period of days or weeks until an effective amount is being administered.

Preferably, the gradual introduction of the compound diminishes the development of a flu-like syndrome. More preferably, the gradual introduction is accomplished by, during the first two months of administration of the compound, the amount of compound administered daily is gradually increased, such that less than about 0.5 mg/kg is administered daily for the first 14 days, and is increased to more than about 2.0 mg/kg daily at the end of two months.

The invention further provides a method for treating a mammal with diabetes mellitus to delay the onset of end stage renal disease, wherein said method comprises administering to said mammal an effective amount of a pharmaceutical composition, wherein said composition comprises a compound selected from the group consisting of aminoguanidine, its pharmaceutically acceptable salts, and mixtures thereof, and wherein the mammal is periodically screened during treatment for crescentic glomerulonephritis.

Preferably, the mammal is screened by measurement of anti-neutrophil cytoplasmic antibodies of the myeloperoxidase type (MPO-ANCAs) levels. Also preferably, the mammal is screened about every three months. Also, in one embodiment, upon a positive screening result, treatment with the composition is stopped.

The invention further provides a method for treating a mammal with diabetes mellitus to delay the onset of end stage renal disease, said method comprising administering to said mammal an effective amount of a pharmaceutical composition, wherein said composition comprises a compound selected from the group consisting of aminoguanidine, its pharmaceutically acceptable salts, and mixtures thereof, and wherein the subject shows indicia of overt diabetic nephropathy. In certain embodiments the subject has a serum creatinine level of about 1.8 mg/dL or less at the time of beginning treatment.

Further objects and advantages of the present invention will be clear from the description that follows.

### DETAILED DESCRIPTION OF THE INVENTION

The invention is directed to, among other things, treatment of a mammal with diabetes mellitus. Diabetes mellitus, or diabetes, is a disease related to a disorder of the pancreatic hormone, insulin, which regulates the uptake of blood glucose. Diabetes occurs in several forms, including Type I (insulin-dependent), Type II (noninsulin-dependent), and gestational. Type I and Type II diabetes can lead to end stage renal disease (ESRD), which is fatal if untreated. Usually, progressive renal histological damage occurs in diabetic patients, which can lead to overt proteinuria, a reduction in the glomerular filtration rate, and eventually, ESRD. ESRD is renal disease sufficiently advanced such that maintenance dialysis or renal transplantation is medically advised. Often in ESRD the kidneys are functioning at approximately ten percent (10%) or less of normal capacity. ESRD can be tracked and diagnosed by a variety of clinical methods, including measurement of the glomerular filtration rate, the iothalamate clearance method and measurement of serum creatinine. Lewis E., Hunsicker, L. Bain, R., et al. “A clinical trial of an angiotensin converting enzyme inhibitor in the nephrophathy of insulin-dependent diabetes mellitus,” NEJM 329: 1456-62, 1993. ESRD is associated with abnormally high levels of serum creatinine, that is, levels in excess of approximately 1.4 mg/dl. Serum creatinine levels can be used to calculate creatinine clearance use the Cockcroft and Gault formula. Cockcroft, D. S. and Gault, M. H., “Prediction of creatinine clearance from serum creatinine,” Nephron, (1976), 16:31-41.

To delay the onset of ESRD by administration of an effective amount of a pharmaceutical compound means a sufficient dose of such compound in a pharmaceutical composition is administered so that, compared with an untreated population, a treated population will survive a longer period of time before suffering from abnormally high levels of serum creatinine. Pharmaceutical compositions can be prepared to allow a therapeutically effective quantity of the compound of the present invention, and can include a pharmaceutically acceptable carrier, selected from known materials utilized for this purpose. See, e.g., Remington, The Science and Practice of Pharmacy, 1995; Handbook of Pharmaceutical Excipients, 3^{rd} Edition, 1999. Such compositions can be prepared in a variety of forms, depending on the method of administration.

The pharmaceutical compound of the present invention, aminoguanidine, and its associated salts, is also known by the generic name of pimagedine, with an empirical formula of CH₆N₄.HCl, with a formula weight of 110.55.

The compounds of the invention are, for instance, administered orally, sublingually, rectally, nasally, vaginally, topically (including the use of a patch or other transdermal delivery device), by pulmonary route by use of an aerosol, or parenterally, including, for example, intramuscularly, subcutaneously, intraperitoneally, intraarterially, intravenously or intrathecally. Administration can be by means of a pump for periodic or continuous delivery. The compounds of the invention are administered alone, or are combined with a pharmaceutically-acceptable carrier or excipient according to standard pharmaceutical practice. For the oral mode of administration, the compounds of the invention are used in the form of tablets, capsules, lozenges, chewing gum, troches, powders, syrups, elixirs, aqueous solutions and suspensions, and the like. In the case of tablets, carriers that are used include lactose, sodium citrate and salts of phosphoric acid. Various disintegrants such as starch, and lubricating agents such as magnesium stearate and talc, are commonly used in tablets. For oral administration in capsule form, useful diluents are lactose and high molecular weight polyethylene glycols.

If desired, certain sweetening and/or flavoring agents are added. For parenteral administration, sterile solutions of the compounds of the invention are usually prepared, and the pHs of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. For pulmonary administration, diluents and/or carriers will be selected to be appropriate to allow the formation of an aerosol. See, Remington's Pharmaceutical Sciences, 16th Ed., Mack Publishing, Easton, Pa., 1980, as well as later editions, for information on pharmaceutical compounding.

Suppository forms of the compounds of the invention are useful for vaginal, urethral and rectal administrations. Such suppositories will generally be constructed of a mixture of substances that is solid at room temperature but melts at body temperature. The substances commonly used to create such vehicles include theobroma oil, glycerinated gelatin, hydrogenated vegetable oils, mixtures of polyethylene glycols of various molecular weight and fatty acid esters of polyethylene glycol. See, Remington's Pharmaceutical Sciences, 16th Ed., Mack Publishing, Easton, PA, 1980, pp. 1530-1533 for further discussion of suppository dosage forms. Analogous gels or creams can be used for vaginal, urethral and rectal administrations, and for other topical administrations.

Numerous administration vehicles will be apparent to those of ordinary skill in the art, including without limitation slow release formulations, liposomal formulations and polymeric matrices.

In a preferred embodiment, the administration is in tablet form, and is administered orally. Tablets can be formulated using a variety of inactive ingredients, including carnauba wax, hydroxypropyl cellulose, colloidal silicon dioxzide, calcium stearate, hydroxypropyl methylcellulose, titantium dioxide, polydextrose, triacetin, polyethyene glycol and synthetic iron oxide. Tablets can be formulated in various dosage strengths, including 50, 100, 200 and 400 mg of aminoguanidine.

In another preferred embodiment, the pharmaceutically effective amount is approximately 4-8 mg/kg body weight daily. Still more preferably, the pharmaceutically effective amount is approximately 2-4 mg/kg body weight daily. In a preferred embodiment, the amount is administered in two equal daily doses, each dose of approximately 1-4 mg/kg body weight.

Another aspect of the invention is periodic screening of the subject for crescentic glomerulonephritis. Crescentic glomerulonephritis, or rapidly progressing glomerulonephritis, is the destruction of glomeruli within the kidneys, characterized by the presence of crescent structures upon biopsy of the kidney, and progressive loss of kidney function in days to weeks. The development of this disease is a rare adverse event associated with treatment with pharmaceutically effective amounts of the compound. Periodic screening refers to screening by a medical professional or home testing on a regular basis. In a preferred embodiment, the subject is screened every six (6) months. Still more preferably, the subject is screened every three (3) months. Screening refers to a check for symptoms of crescentic glomerulonephritis, which can be by physical examination, questioning of the subject, and/or analysis of specimens, including standard urinalysis, measurement of blood urea nitrogen (“BUN”) or serum creatinine levels, measurement of creatinine clearance, testing for anti-glomerular basement membrane antibodies, a kidney biopsy, or measurement of myeloperoxidase-anti-neutrophile cytoplasmic autoantibody (“MPO-ANCA”) levels. In a preferred embodiment, MPO-ANCA levels are measured. In another preferred embodiment, administration of the compound is stopped upon a positive screening result. Still more preferably, administration is stopped upon a MPO-ANCA level higher than approximately 10,000U/ml.

Another aspect of the invention is treating a mammal, particularly a human, who/which has a serum creatinine level of approximately 1.0 mg/dL or more at the time of beginning treatment, or a creatinine clearance rate of 50 ml/min. or less, or a 24-hour protein excretion of 500 mg/day or more. In a preferred embodiment, the serum creatinine level is less than about 1.8 mg/dL.

Administration Protocol

The present methods hold the promise for arresting, and to some extent reversing, the aging of key proteins both in animals and plants, and concomitantly, conferring both economic and medical benefits as a result thereof.

The therapeutic implications of the present invention relate to the arrest, and to some extent, the reversal of the aging process which has, as indicated earlier, been identified and exemplified in the aging of key proteins by advanced glycosylation and cross-linking. Thus, body proteins, and particularly structural body proteins, such as collagen, elastin, lens proteins, nerve proteins, kidney glomerular basement membranes and other extravascular matrix components would all benefit in their longevity and operation from the practice of the present invention. The present invention thus reduces the incidence of pathologies involving the entrapment of proteins by cross-linked target proteins, such as retinopathy, cataracts, diabetic kidney disease, glomerulosclerosis, peripheral vascular disease, arteriosclerosis obliterans, peripheral neuropathy, stroke, hypertension, atherosclerosis, osteoarthritis, periarticular rigidity, loss of elasticity and wrinkling of skin, stiffening of joints, glomerulonephritis, and other conditions. Likewise, all of these conditions are in evidence and tend to occur at an accelerated rate in patients afflicted with diabetes mellitus as a consequence of this hyperglycemia. Thus, the present therapeutic method is relevant to treatment of these and related conditions in patients either of advanced age or those suffering from one of the mentioned pathologies.

Protein cross-linking through advanced glycosylation product formation can decrease solubility of structural proteins such as collagen in vessel walls and can also trap serum proteins, such as lipoproteins to the collagen. Also, this can result in increased permeability of the endothelium and consequently covalent trapping of extravasated plasma proteins in subendothelial matrix, and reduction in susceptibility of both plasma and matrix proteins to physiologic degradation by enzymes. For these reasons, the progressive occlusion of diabetic vessels induced by chronic hyperglycemia is believed to result from excessive formation of glucose-derived cross-links. Such diabetic microvascular changes and microvascular occlusion can be effectively prevented and reversed by chemical inhibition and reversal of the advanced glycosylation product formation utilizing a composition and the methods of the present invention.

Molecular cross-linking through advanced glycosylation product formation can decrease solubility of structural proteins such as collagen in vessel walls and can also trap serum proteins, such as lipoproteins to the collagen. Also, this can result in increased permeability of the endothelium and consequently covalent trapping of extravasated plasma proteins in subendothelial matrix, and reduction in susceptibility of both plasma and matrix proteins to physiologic degradation by enzymes. For these reasons, the progressive occlusion of diabetic vessels induced by chronic hyperglycemia has been hypothesized to result from excessive formation of sugar-derived and particularly, glucose-derived cross-links. Such diabetic microvascular changes and microvascular occlusion can be effectively prevented and reversed by chemical inhibition and reversal of the advanced glycosylation product formation utilizing a composition and the methods of the present invention.

Diabetes-induced changes in the deformability of red blood cells, leading to more rigid cell membranes, is another manifestation of cross-linking and aminoguanidine has been shown to prevent it in vivo. In such studies, New Zealand White rabbits, with induced, long-term diabetes are used to study the effects of a test compound on red blood cell (RBC) deformability. The test compound is administered at a rate of 100 mg/kg by oral gavage (tube delivery to stomach) to diabetic rabbits.

A further consequence of diabetes is the hyperglycemia-induced matrix bone differentiation resulting in decreased bone formation usually associated with chronic diabetes. In animal models, diabetes reduces matrix-induced bone differentiation by 70%.

Methods of the invention can comprise administering aminoguanidine in an effective amount for (i) improving the elasticity or reducing wrinkles of a skin, treating (ii) diabetes or treating or inhibiting the (iii) discoloration of teeth, or treating or preventing one or more of the following conditions: (iv) adverse sequelae of diabetes, (v) kidney damage, (vi) damage to blood vasculature (e.g., stiffening), (vii) hypertension, (viii) retinopathy, (ix) damage to lens proteins, (x) cataracts, (xi) neuropathy or (xii) osteoarthritis, or (xiii) improving myocardial elasticity. Such damage can be damage to intraperitoneal tissue caused by repeated contact with sugar-laden IP dialysis compositions, or damage to vascular tissue caused by contact with sugar-laden dialysis compositions (IP or otherwise).

One aspect of the invention is the gradual introduction of the compound to the mammal (e.g., human) receiving treatment. “Gradual introduction” refers to beginning treatment with a low dose of the compound, and periodically increasing the dose until the pharmaceutically effective amount is reached. In a preferred embodiment, administration is begun at approximately 0.25-0.5 mg/kg, the dosage is increased by approximately 0.5 mg/kg every seven to fourteen days until the targeted pharmaceutically effective amount is reached. The targeted administration amount is typically 1.0 to 2.0 mg/kg/day. Though the schedule can vary, preferably the targeted administration amount is reached within a period of from thirty to sixty days, more preferably twenty-one to forty-two days. Still more preferably, administration is begun at approximately 0.5 mg/kg, increased by approximately 0.5 mg/kg every fourteen days, such that an effective amount is being administered at the end of approximately every two months.

In a preferred embodiment, the gradual introduction is performed such that the development of a flu-like syndrome in the subject receiving treatment is diminished or avoided. As further set forth below, studies have shown that in some subjects, beginning administration of a pharmaceutically effective amount is associated with the development of a flu-like syndrome, which can include lethargy, weakness, fever, chills, myalgia (e.g., pain in one or more muscles), arthralgia (e.g., neuralgic pain in on or more joints), rash, elevated liver enzymes and decreased hematocrit, which syndrome can begin two to six weeks after initiation of treatment and last two to four weeks.

In one embodiment, the invention discloses classes of compounds that can also reverse the cardiovascular stiffness associated with normal aging in mammals. By breaking established A.G.E. cross-links, it is believed that these classes of compounds modify diastolic stiffness associated with the left ventricle. As a result, cardiac function significantly improves, as evidenced by increased left ventricular (LV) end diastolic volume (EDV), stroke volume, and decreased end diastolic pressure (EDP). While it is believed that the method of the present invention is accomplished by the above described mechanism, the possibility that the method of the present invention improves myocardial elasticity through alternative mechanisms is not ruled out.

The present invention provides for methods that can be used to monitor hemodynamic indicators of myocardial elasticity. These indicators can be used to monitoring subjects during the course of therapy of compound administration, and they can also be used to identify patients that are candidates for the method of the present invention. Useful hemodynamic indicators of myocardial elasticity include left 25 ventricular end-diastolic volume (EDV), stroke volume, end-diastolic pressure (EDP), and left ventricular stiffness.

Left ventricular stiffness is a preferred measure of myocardial elasticity. This parameter can be calculated from the end-diastolic volume (EDV) and the end-diastolic pressure (EDP). These two parameters can be determined experimentally in dogs. One method useful for the measurements in dogs involves the introduction of catheters into the left ventricular (LV) chamber and proximal aorta via the carotid artery. Goodale-Lubin catheters (no. 8 French), for example, can be used. Transducers with a suitable physiological recording system using pressure amplifiers and a fluid-filled catheter system optimally damped for frequency-response can record LV and arterial pressures. Typically the transducers are placed at the mid-thoracic level and balanced to provide for equal sensitivity. In animals the invention provides for measurement of EDV and EDP after intravascular volume loading. Typically the increased intravascular loading is accomplished through administration of 10% dextran-40. Preferably the infusion rate of the loading agent is 3 ml-min⁻¹-kg-1 over 3 min. In the intact animal, simultaneous measures of LV pressure and volume can be made before and after volume loading. EDP can be measured from pressure determinations at the end-expiration phase of the respiratory cycle.

LV volume can be determined by two-dimensional echocardiography. In a preferred embodiment, imaging location and time-gain settings are adjusted to yield optimal definition of endocardial borders, which can be delineated by bubbling saline into the LV chamber. Preferably the influence of heart rate on these measurements is minimized, by comparing ventricular dimensions at similar R-R intervals. In a preferred embodiment this comparison is made using an Ultramark 4 system (Advanced Technology Laboratories). The end-diastolic and end-systolic dimensions for three to four consecutive cardiac cycles can be measured and averaged, and the ejection fraction and stroke volume subsequently calculated. Ventricular volume can be derived by the length-diameter method (Vuille, C. & Weyman, A. E. (1994) In Principles and Practice of Echocardiography, ed. Weyman, A. E. (Lea & Gebiger, Philadelphia), pp. 575-624), with apical views for measurements taken from the inner margins of the endocardial echoes. Endocardial and epicardial borders can be traced directly from the video display onto a digitizing tablet. End-diastolic frames can be selected for analysis by using the R wave as a marker for end diastole.

To characterize the diastolic pressure-volume relationship in the left ventricle (Gaasch, W. H. (1994) in Left Ventricular Diastolic Dysfunction and Heart Failure, eds. Gaasch, W. H. & Lewinter, M. M. (Lea & Febiger, Philadelphia), pp. 143-149), the exponential equation P=be^{kV} can be used, where P=pressure in mmHg, V=volume in ml/kg, b=the pressure intercept in mmHg, and k represents the modulus of chamber stiffness in the intact ventricle. Two coordinates of pressure and volume can be used. Typically the early diastolic coordinates consist of the lowest value of diastolic pressure before the mitral valve opens and the end systolic volume. EDP and volume can then be utilized as the second coordinates. The chamber-stiffness constant k is calculated as the slope of the natural logarithm of pressure to volume: In (P)=kV+ln(b). Chamber stiffness is derived from the relation dP/dV=kP. With a progressive increase in volumes calculated, myocardial stiffness would be expected to increase as a preload-dependent phenomenon (Kato, S., Spinale, F. G., Tanaka, R., Johnson, W., Cooper, I. V. G. & Zile, M. R. (1995) Am. J Physicol. 269H863-H868). Myocardial stiffness can be calculated from E=k stress.

In a preferred embodiment subjects for the method of the invention are free of valvular or pericardial disease. Typically this assessment can be determined by echocardiography.

The meaning of “effective amount” will be recognized by clinicians but includes an amount effective to (1) reduce, ameliorate or eliminate one or more symptoms of the disease sought to be treated, (2) induce a pharmacological change relevant to treating the disease sought to be treated, or (3) prevent or lessen the frequency of occurrence of a disease.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority or benefit is also incorporated by reference herein in its entirety in the manner described above for publications and references.

The following example further illustrates the present invention but, of course, should not be construed as in any way limiting its scope.

### EXAMPLE 1

In a multicenter, randomized, double-blind, placebo-controlled study, aminoguanidine was administered to 690 patients suffering from Type I diabetes with overt diabetic nephropathy diagnosed by a 24-hour urine total protein excretion of greater than or equal to 500 mg, and a creatinine clearance rate between 40-90 ml/minute, over approximately a four year period. Renal function was measured every three months using serum creatinine levels, and creatinine clearance rates. In addition, the glomerular filtration rate was measured at entry and every six months. Study participants were treated at one of two dosage levels, (150 mg and 300 mg B.I.D.).

Time to doubling of baseline serum creatinine was measured. Study participants were also treated with standard measures for blood pressure control, and diabetes management, including insulin treatment, diet, and exercise. Serum creatinine levels were made at baseline and randomization visits, at the end of weeks 1-6, the end of month 2, the end of month 3, and then every 3 months for the remainder of the study. “Baseline serum creatinine” was the mean of the levels at the baseline and randomization visits. If the level at any subsequent visit was at least two times the baseline, and was confirmed by a second measurement, the patient was considered to have experienced a doubling of baseline serum creatinine. Serum creatinine levels were determined by a clinical laboratory (SciCor Inc., 8211 SciCor Drive, Indianapolis, Ind. 46214-2985).

Twenty-six percent (26%) of placebo patients experienced a doubling of baseline serum creatinine during the course of the study, compared with 20% of patients in the low dose category and 20% in the high dose category (see Table 1). This corresponds to a twenty-nine percent (29%) reduction in the risk of doubling serum creatinine. In patients with a baseline serum creatinine of less than 1.5 mg/dL, treatment of pimagendine resulted in a sixty-three percent (63%) reduction of the risk of doubling serum creatinine. (Table 2).

TABLE 1
Time to Doubling of Serum Creatinine

| | | | Pimagedine | Pimagedine | Pimagedine |
|---|---|---|---|---|---|
| Parameter | Statistics | Placebo | Low Dose | High Dose | Combined |
| Number of patients | N | 236 | 229 | 225 | 454 |
| randomized | | | | | |
| Number of patients with | n | 61 | 45 | 46 | 91 |
| adjudicated doubling | (%) | (26%) | (20%) | (20%) | (20%) |
| Log rank, test, unstratified | p value | | 0.128 | 0.203 | 0.099 |
| | versus | | | | |
| | placebo | | | | |
| Log rank test, stratified for | p value | | 0.113 | 0.122 | 0.059 |
| baseline serum creatinine | versus | | | | |
| | placebo | | | | |
| Odds ratio | odds ratio | | 0.72 | 0.73 | 0.73 |
| | 95% CI | | (0.46, 1.12) | (0.47, 1.14) | (0.50, 1.06) |

TABLE 2
Time to Doubling of Serum Creatinine - Population with Baseline Serum
Creatinine Less Than 1.5 mg/dL.

| | | | Pimagedine | Pimagedine | Pimagedine |
|---|---|---|---|---|---|
| Parameter | Statistics | Placebo | Low Dose | High Dose | Combined |
| Number of patients | N | 130 | 122 | 115 | 237 |
| randomized | | | | | |
| Number of patients with | n | 22 | 13 | 11 | 24 |
| adjudicated doubling | (%) | (17%) | (11%) | (10%) | (10%) |
| Log rank, test, unstratified | p value | | 0.135 | 0.092 | 0.053 |
| | versus | | | | |
| | placebo | | | | |
| Log rank test, stratified for | p value | | 0.116 | 0.075 | 0.044 |
| baseline serum creatinine | versus | | | | |
| | placebo | | | | |
| Odds ratio | odds ratio | | 0.64 | 0.52 | 0.59 |
| | 95% CI | | (0.30, 1.38) | (0.23, 1.15) | (0.31, 1.11) |

Dosage with aminoguanidine at both the low dose and the high dose resulted in statistically significant reductions in 24 hour urinary protein excretion as well. (FIG. 1). The reduction was generally first observed at the first post-randomization evaluation (at three months) and increased in magnitude over the duration of the study. Aminoguanidine exerted a greater effect on proteinuria in patients with a baseline serum creatinine of less than 1.5 mg/dL. (FIG. 2).

Treatment with aminoguanidine also decreased the incidence of progression of retinopathy from baseline to endpoint, as measured by the progression of three or more steps in the Early Treatment Diabetic Retinopathy Study (ETDRS) scale. (Table 3).

TABLE 3
Progression of Diabetic Retinopathy - Three Step or Greater Change in ETDRS
Score Logistic Regression Model.

| | | | Pimagedine | Pimagedine | Pimagedine |
|---|---|---|---|---|---|
| Parameter | Statistics | Placebo | Low Dose | High Dose | Combined |
| Number of patients | N | 236 | 229 | 225 | 454 |
| randomized | | | | | |
| Number of patients with a | N | 164 | 159 | 144 | 303 |
| baseline and endpoint | | | | | |
| evaluation | | | | | |
| Number and % of patients | n | 28 | 18 | 13 | 31 |
| with a ≧ 3 step progression | (%) | (16%) | (11%) | (8%) | (10%) |
| Logistic regression model | odds ratio | | 0.58 | 0.68 | 0.53 |
| | 95% CI | | (.30, 1.14) | (.47, .99) | (.30, .94) |
| | p value | | 0.112 | 0.044 | 0.030 |

### EXAMPLE 2

| | mg/tablet |
|---|---|
| Compound | 50 |
| Starch | 50 |
| Mannitol | 75 |
| Magnesium stearate | 2 |
| Stearic acid | 5 |

The compound, a portion of the starch and the lactose are combined and wet granulated with starch paste. The wet granulation is placed on trays and allowed to dry overnight at a temperature of 45.degree. C. The dried granulation is comminuted in a comminutor to a particle size of approximately 20 mesh. Magnesium stearate, stearic acid and the balance of the starch are added and the entire mix blended prior to compression on a suitable tablet press. The tablets are compressed at a weight of 232 mg using a 11/32″ punch with a hardness of 4 kg. These tablets will disintegrate within a half hour according to the method described in USP XVI.

What is claimed:

1. A method for treating a mammal to (i) improve the elasticity or reducing wrinkles of a skin, treat (ii) diabetes or treat or inhibit the (iii) discoloration of teeth, or treat or prevent one or more of the following conditions: (iv) adverse sequelae of diabetes, (v) kidney damage, (vi) damage to blood vasculature, (vii) hypertension, (viii) retinopathy, (ix) damage to lens proteins, (x) cataracts, (xi) neuropathy or (xii) osteoarthritis, or (xiii) improve myocardial elasticity, or delay (xiv) the onset of end stage renal disease, the method comprising administering to a subject an effective amount of a pharmaceutical composition, wherein said composition comprises a compound selected from the group consisting of aminoguanidine, its pharmaceutically acceptable salts, and mixtures thereof, wherein the compound is administered by gradual introduction over a period of days or weeks until an effective amount is being administered.

2. The method of claim 1, wherein the gradual introduction of the compound diminishes the development of a flu-like syndrome.

3. The method of claim 1, wherein during about the first two months of administration of the compound, the amount of compound administered daily is gradually increased, such that less than about 0.5 mg/kg is administered daily for the first fourteen days, and is increased to about 2.0 mg/kg or more daily at the end of two months.

4. A method for treating a mammal with diabetes mellitus to delay the onset of end stage renal disease, said method comprising administering to the mammal an effective amount of a pharmaceutical composition, wherein said composition comprises a compound selected from the group consisting of aminoguanidine, its pharmaceutically acceptable salts, and mixtures thereof,
wherein the mammal is periodically screened during treatment for crescentic glomerulonephritis.

5. The method of claim 4, wherein the mammal is a human and is screened by measurement of [MPO-ANCA] levels.

6. The method of claim 5, wherein the mammal is a human and is screened about every three months.

7. The method of claim 4, wherein upon a positive screening result, treatment with the composition is stopped.

8. A method for treating a human with diabetes to delay the onset of end stage renal disease, said method comprising administering to the mammal an effective amount of a pharmaceutical composition, wherein said composition comprises a compound selected from the group consisting of aminoguanidine, its pharmaceutically acceptable salts, and mixtures thereof, wherein the subject shows indicia of overt diabetic nephropathy, and wherein the human has a serum creatinine level of about 1.8 mg/dL or less at the time of beginning treatment.
